(19) United States
(12) Patent Application Publication
(10) Pub. No.: US 2019/0192280 A1
(43) Pub. Date: Jun. 27, 2019

(54) System for Adjusting the Shape of a Breast Implant

(71) Applicant: Biosense Webster (Israel) Ltd., Yokneam (IL)

(72) Inventors: Assaf Govari, Haifa (IL); Yehuda Algawi, Binyamina (IL)

(21) Appl. No.: 15/851,396

(22) Filed: Dec. 21, 2017

(51) Int. Cl. A61F 2/12; G16H 20/40

(52) U.S. Cl. CPC A61F 2/12; G16H 20/40; A61M 5/329; A61F 2250/0002; A61F 2250/0001

(57) ABSTRACT

An implant includes a hollow container and a valve. The hollow container is configured to be implanted in an organ of a patient, and to contain filling material. The valve has first and second position sensors coupled thereto, and is configured to allow passage of the filling material to and from the container, so as to vary a volume of the implant.

### FIELD OF THE INVENTION

The present invention relates generally to medical and aesthetic implants, and particularly to methods and systems for shaping a breast implant.

### BACKGROUND OF THE INVENTION

Various types of implants containing filling material, such as breast implants, are known in the art.

For example, U.S. Patent Application Publication 2010/0114311 describes a valve assembly for a mammary implant having a chamber defined by a flexible membrane. The implant includes a valve and a flexible filling tube, which includes a relatively short semi-rigid tubular structure that extends into the chamber and defines a passageway.

U.S. Pat. No. 5,456,716 describes an elastomeric valve assembly designed for use in an inflatable surgical implant to provide a self-sealing means for filling the implant. The valve assembly incorporates vulcanized elastomeric strips molded between two larger silicone sheets, wherein the strips form a collapsible self-sealing channel through which a fill needle may be inserted through slits in the strips and sheets.

### SUMMARY OF THE INVENTION

An embodiment of the present invention that is described herein provides an implant including a hollow container and a valve. The hollow container is configured to be implanted in an organ of a patient, and to contain filling material. The valve has first and second position sensors coupled thereto, and is configured to allow passage of the filling material to and from the container, so as to vary a volume of the implant.

In some embodiments, the valve is configured to allow passage of a syringe therethrough, so as to allow the passage of the filling material to and from the container using the syringe. In other embodiments, the first and second position sensors are configured to produce first and second signals indicative of first and second respective positions of the first and second sensors in a coordinate system of a position tracking system. In yet other embodiments, the hollow container includes an inner hollow container and an outer hollow container disposed around the inner hollow container.

In an embodiment, the inner and outer hollow containers are coupled to the valve at first and second respective positions located at predefined respective first and second distances from the first and second position sensors. In another embodiment, the valve is configured to seal the outer hollow container. In yet another embodiment, the valve is configured to (i) allow passage of a syringe therethrough, so as to allow the passage of the filling material to and from the inner hollow container, and (ii) when no syringe is being passed therethrough, block the passage of the filling material through the inner hollow container.

In some embodiments, the hollow container includes a flexible shell configured to contain the filling material. In other embodiments, the filling material includes at least one of silicone gel and saline solution. In yet other embodiments, the implant includes circuitry, which is configured to receive, from the first and second position sensors, signals indicative of first and second positions of the first and second position sensors, and to transmit an output signal indicative of the first and second positions.

In an embodiment, the circuitry is configured to wirelessly receive electrical power from a device external to the patient. In another embodiment, the implant includes a power source disposed inside the hollow container and configured to be charged wirelessly from a device external to the patient and to provide electrical power to the first and second position sensors.

There is additionally provided, in accordance with an embodiment of the present invention, a system for shaping an implant, the system includes a receiver and a processor. The receiver is configured to receive (i) a first signal indicative of respective positions of one or more position sensors coupled to a valve, which allows passage of filling material to and from the implant, and (ii) a second signal indicative of a position of a position sensor coupled to a syringe that is used, when inserted into the valve, for injecting or extracting the filling material. The processor is configured to calculate and display to a user, based on the first signal and the second signal, an indication of alignment between the syringe and the valve.

In some embodiments, the receiver is configured to receive at least one of the first and second signals wirelessly. In other embodiments, the processor is configured to detect that a misalignment between the syringe and the valve is above a predefined threshold level, and in response to issue a warning.

There is further provided, in accordance with an embodiment of the present invention, a method for shaping an implant, the method includes receiving a first signal indicative of respective positions of one or more position sensors coupled to a valve, which allows passage of filling material to and from the implant. A second signal, which is received, is indicative of a position of a position sensor coupled to a syringe that is used, when inserted into the valve, for injecting or extracting the filling material. Based on the first signal and the second signal, an indication of alignment between the syringe and the valve is calculated and displayed to a user.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a system for shaping a breast implant, in accordance with embodiments of the present invention;

FIG. 2 is a sectional isometric-view of a breast implant, in accordance with embodiments of the present invention;

FIG. 3 is a sectional side-view of a valve of a breast implant, in accordance with embodiments of the present invention; and

FIG. 4 is a flow chart that schematically illustrates a method for shaping an implanted breast implant, in accordance with an embodiment of the present invention.

### DETAILED DESCRIPTION OF EMBODIMENTS

### Overview

Breast implants are prostheses, typically used for reconstructing a human breast after excision, or for shaping the size and contour of breasts in cosmetic applications. A breast implant typically comprises a filling material, also known as implantable material, such as silicone gel that conforms to the texture of natural tissue of the breast.

A typical breast implant further comprises a biocompatible shell adapted to encapsulate the implantable material and to be implanted in the human breast so as to resemble the texture of the breast tissue. The shell typically comprises a soft and flexible material that has no physical or chemical interactions with the surrounding tissue. In some cases, there might be a need or desire to adjust the shape, i.e., the size and contour of the breast implant after the implantation.

Embodiments of the present invention that are described herein provide adjustable-shape breast implants, and systems for adjusting the shape of an implanted breast implant. In some embodiments, a breast implant comprises inner and outer hollow shells that are adapted to contain a suitable filling material. The outer shell is typically filled with silicone gel, whereas the inner shell is filled with a saline solution, referred to herein as “filling material (FM)”.

In some embodiments, the implant comprises a valve adapted to (i) seal the outer shell and (ii) allow passage of a syringe configured to inject or extract FM to or from the inner shell, so as to shape (e.g., vary the volume of) the breast implant.

In some embodiments, the valve comprises outer and inner fasteners located, respectively, at the outer and inner ends of the valve. The outer fastener is coupled to the outer shell of the implant, and the inner fastener is coupled to the inner shell of the implant.

In some embodiments, two position sensors of a position tracking system are coupled to the valve. An outer position sensor is coupled adjacent to the outer fastener and an inner position sensor is coupled adjacent to the inner fastener.

In some embodiments, a user (the patient or another person) adjusts the shape of the breast implant by inserting a syringe into the valve, so as to exchange (e.g., inject and/or extract) some FM with the inner shell. In some embodiments, an additional position sensor, referred to herein as a syringe position sensor, is coupled to the distal end of the syringe.

In some embodiments, the system comprises a processor and an interface. The interface is configured to receive signals indicative of the positions of the outer and inner position sensors of the valve, and of the position of the syringe position sensor. The positions of the sensors are measured in the coordinate system of the position tracking system. In an embodiment, the processor is configured to calculate, based on the received signals, an indication of the alignment between the syringe and the valve, and to display the indication on a suitable display device coupled to the processor.

In some embodiments, the user may navigate the distal end of the syringe, through the valve and into the inner shell, based on the displayed alignment indication. Subsequently, the user may inject FM to, or extract FM from, the inner shell so as to vary the size and contour of the breast implant.

In the context of the present disclosure and in the claims, the terms “shape,” “size” and “volume” are used interchangeably and refer to the shape of the breast implant implanted in the breast of the patient.

The disclosed techniques enable controlling the shape of the breast implant using a procedure that may be carried out by the patient herself, e.g., at home, or by a physician or a nurse at a medical facility or at any other suitable location.

### System Description

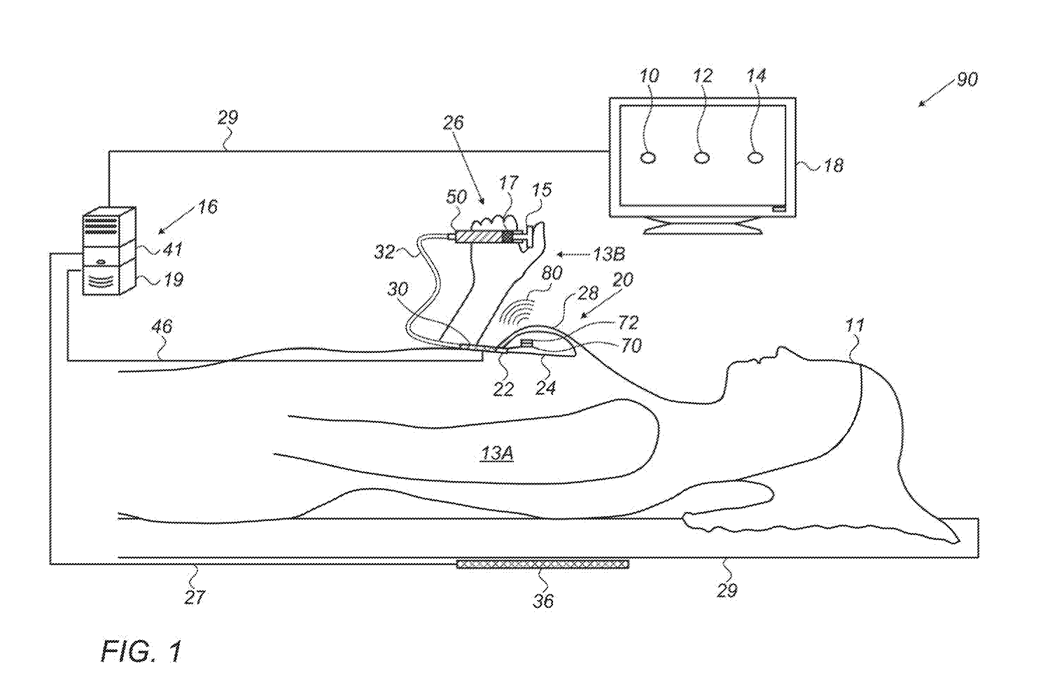

FIG. 1 is a schematic, pictorial illustration of a system 90 for shaping a breast implant 20 implanted in a breast of a patient 11, in accordance with embodiments of the present invention. In some embodiments, system 90 comprises implant 20, which is a prosthesis having an adjustable-shape implanted in the patient breast having natural tissue 28 surrounding implant 20. The implanted prosthesis thus shapes the size and contour of the patient breast.

In some embodiments, implant 20 comprises a hollow outer shell 24 configured to encapsulate one or more types of soft filling material that resemble the texture of tissue 28. In some embodiments, shell 24 physically isolates between the filling material and tissue 28. The filling material is adapted to shape the size and contour of breast implant 20.

In the context of the present disclosure and in the claims, the terms “shell” and “container” are used interchangeably and refer to a hollow, typically flexible, implantable prosthesis configured to contain any suitable filling material, so as to shape the patient breast.

In some embodiments, implant 20 comprises a valve 22, which is configured to allow passage of the filling material to and from implant 20, so as to control the volume of implant 20.

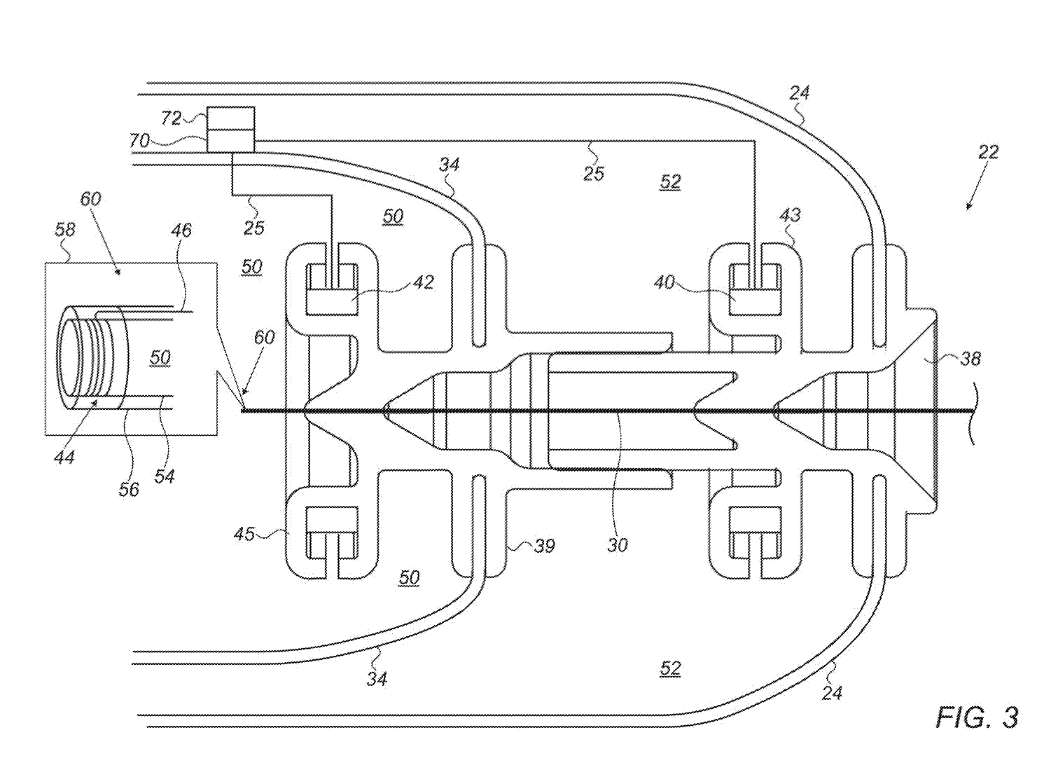

In some embodiments, implant 20 further comprises a battery 70 or any other suitable power source, such as electrical circuitry or a capacitor (not shown) configured to be charged wirelessly. In some embodiments, implant 20 comprises communications circuitry 72, which is configured to wirelessly transmit radio-frequency (RF) signals 80 to a computer 16. In some embodiments, RF signals 80 modulate current levels sensed by one or more position sensors that are fitted on valve 22 and shown in FIG. 3 below.

In some embodiments, system 90 comprises a syringe 26, which is configured to exchange (e.g., inject to implant 20, or extract from implant 20) any suitable fluid of filling material (FM) 50, such as a saline solution, with an internal volume of implant 20. In some embodiments, syringe 26 comprises a needle 30 configured to be inserted, through tissue 28 and valve 22, into implant 20 so as to inject FM 50 to, or to extract FM 50 from, implant 20.

In some embodiments, syringe 26 comprises a barrel 17, a plunger 15, and a flexible filling tube 32 coupled between barrel 17 and needle 30. Barrel 17 is adapted to contain FM 50, and plunger 15 is configured to inject FM 50 to, or extract FM 50 from, implant 20, via flexible filling tube 32.

In some embodiments, a position sensor (shown in FIG. 3 below) of the position tracking system is coupled to the distal tip of needle 30, and is configured to send, via a cable 46, electrical signals indicative of the position of the distal tip of needle 30 in the coordinate system of the position tracking system.

In some embodiments, the position of valve 22 and the distal tip of needle 30 in the heart cavity are typically measured using position sensing techniques. This method of position sensing is implemented, for example, in the CARTO™ system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1, whose disclosures are all incorporated herein by reference.

In some embodiments, computer 16 comprises a driver circuit 41, which drives, via a cable 27, magnetic field generators (not shown) of a location pad 36 placed at a known position external to patient 11 lying on a table 29, e.g., below the patient torso.

In some embodiments, computer 16 comprises a processor 19 having suitable front end and interface circuits for receiving signals from circuitry 72 and needle 30, and for displaying, on a display 18, information of components of system 90, as will be described below.

In some embodiments, processor 19 typically comprises a general-purpose processor, which is programmed in software to carry out the functions described herein. The software may be downloaded to the computer in electronic form, over a network, for example, or it may, alternatively or additionally, be provided and/or stored on non-transitory tangible media, such as magnetic, optical, or electronic memory.

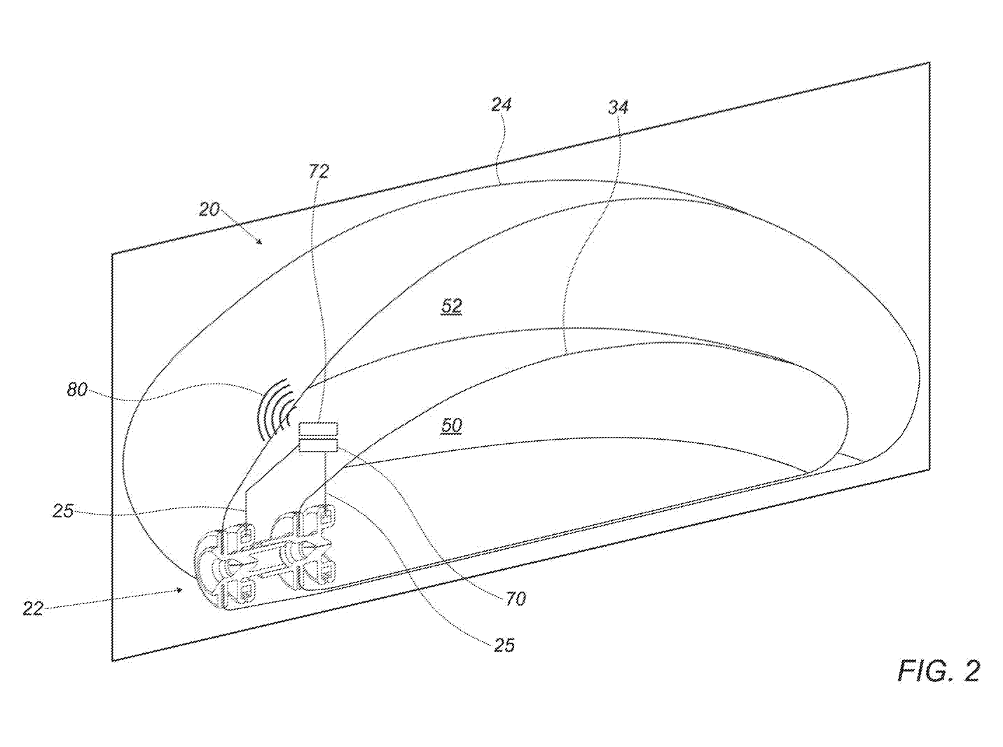

Implant 20, valve 22 and syringe 26 are depicted in detail in FIGS. 2 and 3 below.

In some embodiments, the injection and extraction of FM 50 may be carried out by patient 11 herself, e.g., at home, or by a physician or a nurse, e.g., at a medical facility. In the example of FIG. 1, patient 11 conducts the procedure at home, e.g., by inserting needle 30 using one hand, and injecting FM 50 using the other hand. We generally assume that patient 11 inserts needle 30 using her left hand 13A, and injects FM 50 using her right hand 13B (as shown in FIG. 1) but patient 11 may alternatively insert needle 30 using her right hand 13B and inject FM 50 using her left hand 13A. In other embodiments, patient 11 may insert the syringe and inject FM 50 to, or extract FM 50 FROM, IMPLANT 20, in any other suitable manner.

In some embodiments, processor 19 is coupled to display 18 via a cable 29. The processor is configured to display on display 18, markers 10 and 12 indicating the position of the two position sensors coupled to valve 22, and a marker 14, indicating the position of the distal tip of needle 30. In some embodiments, markers 10, 12 and 14 provide patient 11 with an indication of an alignment level between valve 22 and the distal tip of syringe 30. In some embodiments, processor 19 is configured to display markers 10, 12 and 14 in a common coordinate system so that the user is able to evaluate the positions of the respective sensors relative to one another.

In other embodiments, markers 10, 12 and 14 may be displayed on a hand-held device (not shown), such as a mobile phone or any other device that may receive the relative positions of markers 10, 12 and 14 wirelessly, or via a wire.

In some embodiments, location pad 36 may be located under the torso of patient 11, as shown in FIG. 1. In alternative embodiments, patient 11 may hold location pad 36 below her implanted breast during the insertion of needle 30 into implant 20, and subsequently, may inject FM 50 into implant 20.

In some embodiments, patient 11 may use both hands 13A and 13B to extract some FM 50 from implant 20, for example, by holding barrel 17 using one hand, and pulling plunger 15 using the other hand.

The configuration of system 90 shown in FIG. 1 is an example configuration that is shown purely for the sake of conceptual clarity. In alternative embodiments, any other suitable configuration can be used. For example, any other suitable power source, such as an alternating current (AC) voltage source may be used instead of battery 70.

In some embodiments, circuitry 72 is configured to charge battery 70 (or any other device, such as a capacitor) using RF signals (not shown) received wirelessly from an external unit (not shown) so that battery 70 may power circuitry 72 and the position sensors of valve 22.

### Adjusting the Shape of a Breast Implant

FIG. 2 is a sectional isometric-view of breast implant 20, in accordance with embodiments of the present invention. In some embodiments, breast implant 20 comprises a flexible inner shell 34 and flexible outer shell 24 coupled to one another by valve 22. In some embodiments, the arrangement of shells 24 and 34 forms an outer volume between outer shell 34 and inner shell 34. The outer shell is sealed by valve 22 and the outer volume is filled with a soft filling material that resembles the texture of tissue 28, such as silicone gel 52.

In some embodiments, an inner volume filled with FM 50 is formed within inner shell 34. As described above, the inner volume may be filled with any suitable filling material, such as a saline solution. In this configuration, the amount of material filling the inner volume within shell 34 determines the size and shape of implant 20.

In some embodiments, valve 22 is configured to allow passage of FM 50, via needle 30, to and from inner shell 34, so as to control the amount of FM 50 within inner volume of implant 20. Note that in this configuration, gel 52 is sealed within the outer volume of implant 20. In an embodiment, FM 50 can be injected to, or extracted from, the inner volume only when the distal end of needle 30 is inserted through valve 22, into the inner volume of implant 20.

In some embodiment, valve 22 has a funnel-shaped outer edge (shown in FIG. 2) so as to lead needle 30 conveniently into valve 22.

In some embodiments, battery 70 is electrically connected, via wires 25, to the position sensors (shown in FIG. 3 below) of valve 22. In an embodiment, circuitry 72 is electrically coupled to battery 70 using any suitable coupling or packaging technique.

In some embodiments, battery 70 and circuitry 72 are disposed within the outer volume of implant 20, for example, coupled to an outer surface of inner shell 34 at close proximity to valve 22, as shown in FIG. 2.

In other embodiments, battery 70 and circuitry 72 may be disposed at any other suitable location in implant 20, such as within the internal volume of implant 20. Note that battery 70 and circuitry 72 may be packaged together (e.g., to reduce their combined volume) or disposed as two separate components at two different respective locations within implant 20.

The configuration of valve 22, battery 70 and circuitry 72 are depicted by way of example, and any other suitable configurations can also be used to comply with medical, aesthetic and/or technical requirements. For example, disposing circuitry 72 as close as possible to outer shell 24 may reduce the operational power consumption of circuitry 72 by reducing the thickness of the medium (e.g., gel 52) through which RF signals 80 traverse between circuitry 72 and computer 16. However, it is also desired to minimize the length of wires 25 and to maintain the uniform external texture of implant 20, so that in another configuration, battery 70 and circuitry 72 may be physically coupled to valve 22.

FIG. 3 is a sectional side-view of valve 22, in accordance with embodiments of the present invention. In some embodiments, valve 22 comprises a funnel-shaped outer fastener 38, which is configured to fasten outer shell 24 to valve 22. As described in FIG. 2 above, the funnel shape of fastener 38 assists in leading a distal end 60 of needle 30 into valve 22.

In some embodiments, valve 22 comprises an inner fastener 39, which is configured to fasten inner shell 34 to valve 22.

In some embodiments, valve 22 comprises an outer housing 43 and an inner housing 45, which are configured to contain an outer position sensor 40 and an inner position sensor 42, respectively. In some embodiments, position sensors 40 and 42 may be single axis sensors (SAS), each of them made from a single coil. In alternative embodiments, at least one sensor among sensors 40 and 42 may comprise multiple coils, e.g., three coils, so as to form a three-axis sensor. This configuration may provide the user of system 90 with multi-dimensional positioning, but typically consumes more (e.g., triple) power from battery 70.

In some embodiments, battery 70 and circuitry 72 are coupled to one another and attached to inner shell 34. Note that wires 25 are electrically connecting between each of sensors 40 and 42, and battery 70. In an embodiment, wires 25 are further configured to conduct signals, indicative of the position of position sensors 40 and 42, to circuitry 72. In another embodiment, sensors 40 and 42 may be electrically connected to circuitry 72 using another set of electrical wires (not shown).

Reference is now made to an inset 58. In some embodiments, distal end 60 of needle 30 comprises an outer tube 56 disposed (e.g., coaxially) around an inner tube 54. In an embodiment, outer tube 56 is configured to puncture the skin and tissue 28 of patient (or any soft container) so as to enable contact between inner tube 54 and valve 22. In another embodiment, the puncturing of the patient skin may be carried out using a puncturing shaft threaded through inner tube 54 for puncturing and retracted out of needle 30 after puncturing, or using any other suitable puncturing technique.

In some embodiments, a single coil is wrapped around the distal tip of inner tube 54, so as to serve as a single-axis position sensor 44. In some embodiments, sensor 44 is electrically coupled to processor 19, via cable 46 that is threaded along needle 30 between inner tube 54 and outer tube 56. In other embodiments, cable 46 may be printed, for example, on the outer surface of inner tube 54.

In these embodiments, cable 46 may comprise multiple wires, such that one or more wires provide power supply from computer 16 to sensor 44, and one or more other wires of cable 46 may conduct, between sensor 44 and processor 19, electrical signals indicative of the position of sensor 44.

In other embodiments, position sensor 44 may comprise multiple (e.g., three) coils so as to form a three-axis position sensor (TAS). In these embodiments, power consumption is received from computer 16 so that power consumption by sensor 44 is not limiting the operation of system 90. In this configuration the TAS (not shown) is typically disposed between tubes 54 and 56, so as to enable free passage of FM 50 through tube 54.

In these embodiments, sensor 44 may comprise a flat multi-axis sensor (e.g., TAS) printed, for example, on a flexible printed circuit board (PCB) wrapped around inner tube 54. In an embodiment, such a TAS is depicted, for example, in U.S. patent application Ser. No. 15/433,072, filed Feb. 15, 2017, which is incorporated herein by reference.

Reference is now made to FIG. 1. In some embodiments, during the injection procedure, a receiver (e.g., interface circuits) of processor 19, is configured to receive from circuitry 72, signals 80 indicative of the position of sensors 40 and 42 coupled to valve 22, and from needle 30 signals indicative of the position of sensor 44 coupled to distal end 60.

In these embodiments, processor 19 is configured to display to patient 11 (or to any other user of system 90) on display 18, markers 10 and 12, which are indicative of the respective positions of the inner and outer housings of valve 22. In some embodiments, patient 11 may navigate needle 30 through valve 22, based on the displayed alignment between markers 10 and 12 indicating the position of valve 22, and marker 14 indicating the position of distal end 60.

In the example of FIG. 1, marker 14 indicates that distal end 60 of needle 30 passed through valve 22, so that patient 11 may stop the insertion of needle 30 and inject FM 50 into the inner volume of implant 20.

In some embodiments, processor 19 is configured to issue a warning signal in case the distance between sensors 14 and 12, or the distance between sensor 14 and 10, exceed a predefined distance. This warning signal indicates to the operator of system 90 (e.g., patient 11) that distal end 60 is either not inserted into valve 22 (sensed by exceeded distance between sensors 10 and 14), or inserted too deep into the internal volume of shell 24 (sensed by exceeded distance between sensors 12 and 14), thereby risking a puncture of shell 24 by needle 30.

In other embodiments, an RF transmitter (not shown) may be coupled to needle 60 and electrically coupled to computer 16, or to any external power source. In these embodiments, the RF transmitter is configured to wirelessly charge battery 70 (or the capacitor described above) with electrical power, so that battery 70 (or the capacitor) may power circuitry 72 and position sensors 40 and 42 of valve 22. In an embodiment, the RF transmitter may be coupled to the distal end of inner tube 54 and may receive power via cable 46 or via a dedicated cable coupled to computer 16 or to any other suitable external power source.

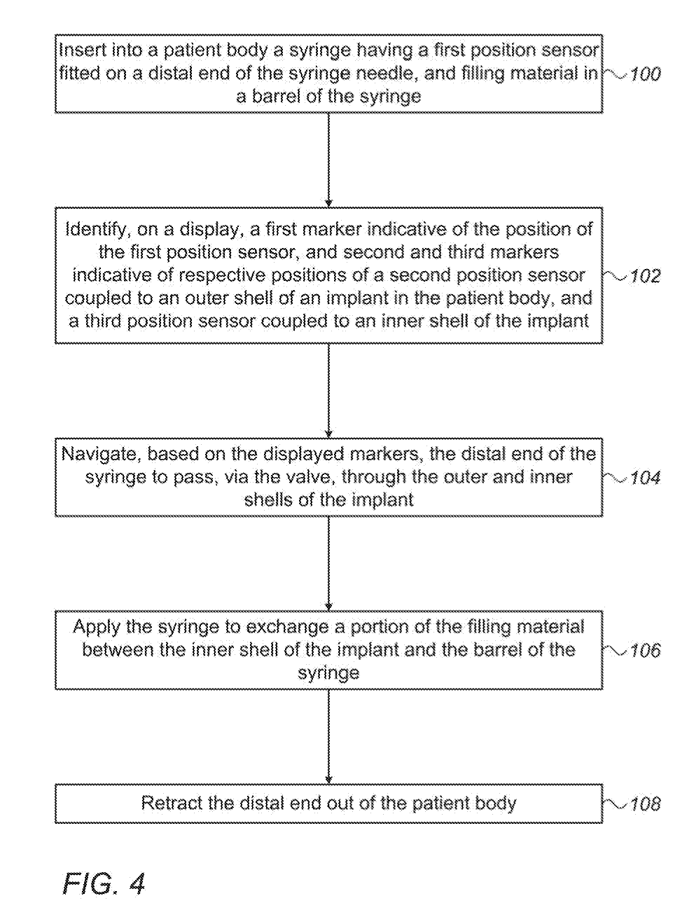

FIG. 4 is a flow chart that schematically illustrates a method for shaping breast implant 20, in accordance with an embodiment of the present invention.

The method begins with patient 11, or any other user of system 90, inserting needle 30 into the breast of patient 11, at a needle insertion step 100. In some embodiments, position sensor 44 is coupled to needle 30 of syringe 26, which is configured to exchange FM 50 between barrel 17 and the internal volume of implanted implant 20.

In some embodiments, patient 11 may have location pad 36 placed at a known position external to her body, e.g., below her torso, as depicted in FIG. 1 above. In other embodiments, during the insertion of needle 30 into her implanted breast, patient 11 may hold location pad 36 below her implanted breast, e.g., using one of her hands, and insert needle 30 using her other hand. In these embodiments, patient 11 may sit or stand so as to enable the positioning of location pad 36 below her implanted breast.

At a markers identification step 102, patient 11 identifies on display 18, markers 10 and 12 indicating respective positions of outer housing 43 and inner housing 45 of valve 22, and further identifies marker 14 indicative of the position of distal end 60.

At a navigation step 104, based on the locations of markers 10, 12 and 14, patient 11 navigates distal end 60 to pass, via valve 22, through outer shell 24 and inner shell 34 of implant 20, so as to insert distal end 60 into the inner volume of implant 20. In some embodiments, navigation step 104 is concluded after patient 11 verifies, on display 18, that distal end 60 is positioned at the inner volume of implant 20.

At an injection step 106, patient 11 injects FM 50 from barrel 17 into the inner volume of implant 20, so as to increase the volume of implant 20. In some embodiments, after injecting FM 50 into implant 20, patient 11 may check the size of her implanted breast. Based on the volume of the implanted breast, patient 11 may inject additional FM 50 into implant 20, or alternatively, may extract some FM 50 from implant 20 into barrel 17, so as to reduce the volume of the implanted breast.

At a needle extraction step 108, after obtaining the desired volume of the implanted breast, patient may retract needle 30 out of valve 22 while tracking the position of marker 14 relative to the positions of markers 10 and 12, and may conclude the method after retracting needle 30 out of her breast.

As described above, the method depicted in FIG. 4 may be carried out by patient 11 herself at home, or alternatively, may be carried out by a physician or a nurse at a clinical facility.

Although the embodiments described herein mainly address breast implants, the methods and systems described herein can also be used in other applications, such as in any shape-controlled implantable device.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. An implant, comprising:
a hollow container, which is configured to be implanted in an organ of a patient, and to contain filling material; and
a valve, which has first and second position sensors coupled thereto, and which is configured to allow passage of the filling material to and from the container, so as to vary a volume of the implant.

2. The implant according to claim 1, wherein the valve is configured to allow passage of a syringe therethrough, so as to allow the passage of the filling material to and from the container using the syringe.

3. The implant according to claim 2, wherein the first and second position sensors are configured to produce first and second signals indicative of first and second respective positions of the first and second sensors in a coordinate system of a position tracking system.

4. The implant according to claim 1, wherein the hollow container comprises an inner hollow container and an outer hollow container disposed around the inner hollow container.

5. The implant according to claim 4, wherein the inner and outer hollow containers are coupled to the valve at first and second respective positions located at predefined respective first and second distances from the first and second position sensors.

6. The implant according to claim 4, wherein the valve is configured to seal the outer hollow container.

7. The implant according to claim 4, wherein the valve is configured to (i) allow passage of a syringe therethrough, so as to allow the passage of the filling material to and from the inner hollow container, and (ii) when no syringe is being passed therethrough, block the passage of the filling material through the inner hollow container.

8. The implant according to claim 1, wherein the hollow container comprises a flexible shell configured to contain the filling material.

9. The implant according to claim 1, wherein the filling material comprises at least one of silicone gel and saline solution.

10. The implant according to claim 1, and comprising circuitry, which is configured to receive, from the first and second position sensors, signals indicative of first and second positions of the first and second position sensors, and to transmit an output signal indicative of the first and second positions.

11. The implant according to claim 10, wherein the circuitry is configured to wirelessly receive electrical power from a device external to the patient.

12. The implant according to claim 1, and comprising a power source disposed inside the hollow container and configured to be charged wirelessly from a device external to the patient and to provide electrical power to the first and second position sensors.

13. A system for shaping an implant, the system comprising:
a receiver, configured to receive (i) a first signal indicative of respective positions of one or more position sensors coupled to a valve, which allows passage of filling material to and from the implant, and (ii) a second signal indicative of a position of a position sensor coupled to a syringe that is used, when inserted into the valve, for injecting or extracting the filling material; and
a processor, configured to calculate and display to a user, based on the first signal and the second signal, an indication of alignment between the syringe and the valve.

14. The system according to claim 13, wherein the receiver is configured to receive at least one of the first and second signals wirelessly.

15. The system according to claim 13, wherein the processor is configured to detect that a misalignment between the syringe and the valve is above a predefined threshold level, and in response to issue a warning.

16. A method for shaping an implant, the method comprising:
receiving a first signal indicative of respective positions of one or more position sensors coupled to a valve, which allows passage of filling material to and from the implant;
receiving a second signal indicative of a position of a position sensor coupled to a syringe that is used, when inserted into the valve, for injecting or extracting the filling material; and
calculating and displaying to a user, based on the first signal and the second signal, an indication of alignment between the syringe and the valve.

17. The method according to claim 16, wherein receiving the first and second signals comprises receiving at least one of the first and second signals wirelessly.

18. The method according to claim 16, and comprising detecting that a misalignment between the syringe and the valve is above a predefined threshold level, and in response issuing a warning.

19. A syringe needle, comprising:
a first hollow tube, which is coupled to a barrel of a syringe and is configured to exchange fluid between the barrel and a container into which the syringe needle is inserted;
a second hollow tube, disposed around the first hollow tube; and
a position sensor, disposed at a predefined location between the first and second hollow tubes, and configured to produce a signal indicative of a position of the predefined location in a coordinate system of a position tracking system.

20. The syringe needle according to claim 19, and comprising a cable, which passes between the first and second hollow tubes, and which is configured to conduct electrical signals between the position sensor and the position tracking system.
